(19) United States
(12) Patent Application Publication
(10) Pub. No.: US 2025/0302428 A1
(43) Pub. Date: Oct. 2, 2025

(54) ULTRASOUND IMAGING METHOD FOR ASSISTING IN DETERMINING CARDIAC REGURGITATION, AND ULTRASOUND IMAGING APPARATUS

(71) Applicant: GE Precision Healthcare LLC, Waukesha, WI (US)

(72) Inventors: Yao Ding, Wuxi (CN); Hongjian Jiang, Wuxi (CN); Yizhen Zhang, Wuxi (CN); Zhiqiang Jiang, Wuxi (CN)

(21) Appl. No.: 19/092,782

(22) Filed: Mar. 27, 2025

(30) Foreign Application Priority Data
Mar. 29, 2024 (CN) 202410381083.7

(51) Int. Cl. A61B 8/06; A61B 8/00; A61B 8/08

(52) U.S. Cl. CPC A61B 8/065; A61B 8/0883; A61B 8/463; A61B 8/488; A61B 8/54

(57) ABSTRACT

An ultrasound imaging method for assisting in determining cardiac regurgitation and an ultrasound imaging apparatus. The method includes: performing first ultrasound imaging on a heart, to generate a two-dimensional color flow image of the heart; automatically identifying suspected regurgitation in the two-dimensional color flow image; and automatically performing second ultrasound imaging based on an identification result of the suspected regurgitation, to generate a second ultrasound image containing a suspected regurgitation region, the second ultrasound image being different from the two-dimensional color flow image and containing blood flow information. According to the embodiments of the present application, the workload of regurgitation detection by a physician is reduced, and the accuracy of regurgitation detection is improved.

### CROSS-REFERENCE TO RELATED APPLICATION

This application claim priority to Chinese Patent Application No. 202410381083.7, which was file on Mar. 29, 2024 at the Chinese Patent Office. The entire contents of the above-listed application are incorporated by reference herein in their entirety.

### TECHNICAL FIELD

Embodiments of the present application relate to the technical field of medical imaging, and relate in particular to an ultrasound imaging method for assisting in determining cardiac regurgitation and an ultrasound imaging apparatus.

### BACKGROUND

Cardiac regurgitation is a cardiac abnormality. Normally, heart valves (e.g., the mitral valve and the tricuspid valve) are completely closed before ventricles contract to pump blood, to ensure that all the blood in the ventricles is ejected into the aorta. If the valves are not completely closed, a portion of the blood in the ventricles will be ejected back into atria through openings in the incompletely sealed valves. Cardiac regurgitation reduces blood flows flowing from the heart and reduces the efficiency of cardiac contraction. In this case, the heart will increase its pumping rate to maintain blood supply, and if this continues, heart failure is likely to occur.

A physician may image the heart by means of an ultrasound imaging system to detect the phenomenon of cardiac regurgitation. First, a two-dimensional color image of the heart is obtained by the ultrasound imaging system, and the physician determines, according to the two-dimensional color image, a location where heart regurgitation is occurring. In addition, the physician may alternatively use another imaging mode to detect cardiac regurgitation, for example, use an ultrasound imaging mode containing blood flow information, such as a continuous wave Doppler mode, a pulsed wave Doppler mode, or a four-dimensional color ultrasound mode, to determine whether cardiac regurgitation is occurring.

It should be noted that the above introduction of the background is only for the convenience of clearly and completely describing the technical solutions of the present application, and for the convenience of understanding for those skilled in the art.

### SUMMARY

The inventors of the present application have found that existing solutions for determining cardiac regurgitation have the following drawbacks: a location (e.g., a hole) of a pathological defect in a heart valve that leads to cardiac regurgitation is usually very small. During screening using a color flow imaging mode, it is difficult for a two-dimensional section on which a two-dimensional color image is located to pass through a corresponding hole. In addition, even if the two-dimensional section of the two-dimensional color image passes through the corresponding hole, a signal of a regurgitation region is relatively weak compared with the entire color image, and a corresponding blood flow characteristic may be missed. A weak blood flow signal also introduces a significant possibility of misjudgment in determining regurgitation. In this case, a physician cannot be certain whether regurgitation has occurred. Although the physician may switch the imaging mode to assist in determining whether regurgitation in the two-dimensional color image is true, a switching process is highly likely to cause a previously noted region where regurgitation may exist to disappear from an ultrasound image once a probe is inadvertently moved.

To address at least one of the foregoing problems or other similar problems, provided in embodiments of the present application are an ultrasound imaging method for assisting in determining cardiac regurgitation and an ultrasound imaging apparatus.

According to an aspect of the embodiments of the present application, an ultrasound imaging method for assisting in determining cardiac regurgitation is provided. The method comprises:
performing first ultrasound imaging on a heart, to generate a two-dimensional color flow image of the heart;
automatically identifying suspected regurgitation in the two-dimensional color flow image; and
automatically performing second ultrasound imaging based on an identification result of the suspected regurgitation, to generate a second ultrasound image containing a suspected regurgitation region, the second ultrasound image being different from the two-dimensional color flow image and containing blood flow information.

In one or more embodiments, the automatically identifying suspected regurgitation in the two-dimensional color flow image comprises:
automatically identifying occurrence of the suspected regurgitation in the two-dimensional color flow image, and automatically determining a location of the suspected regurgitation.

In one or more embodiments, the automatically performing second ultrasound imaging based on an identification result of the suspected regurgitation comprises:
automatically controlling, based on the determined location of the suspected regurgitation, an imaging parameter of the second ultrasound imaging, to correlate the second ultrasound imaging with the location of the suspected regurgitation.

In one or more embodiments, the second ultrasound imaging comprises at least one of continuous wave Doppler imaging, pulsed wave Doppler imaging, and four-dimensional color ultrasound imaging.

In one or more embodiments, the second ultrasound imaging comprises continuous wave Doppler imaging or pulsed wave Doppler imaging; and the automatically controlling an imaging parameter of the second ultrasound imaging comprises:
automatically positioning, based on the location of the suspected regurgitation, a location of a sampling window of the second ultrasound imaging, so that the sampling window passes through the suspected regurgitation region; and automatically performing the second ultrasound imaging on the location of the sampling window, to generate a continuous wave Doppler image or a pulsed wave Doppler image related to the suspected regurgitation region.

In one or more embodiments, the second ultrasound imaging comprises four-dimensional color ultrasound imaging; and the automatically controlling an imaging parameter of the second ultrasound imaging comprises:
generating a four-dimensional color ultrasound image of the heart, mapping the location of the suspected regurgitation in the two-dimensional color flow image into the four-dimensional color ultrasound image, and highlighting the location.

In one or more embodiments, the method further comprises:
simultaneously displaying the two-dimensional color flow image, the location of the suspected regurgitation in the two-dimensional color flow image, the four-dimensional color ultrasound image, and the location of the suspected regurgitation in the four-dimensional color ultrasound image.

In one or more embodiments, the automatically identifying suspected regurgitation in the two-dimensional color flow image further comprises:
automatically determining the time of the suspected regurgitation and predicting the time of at least one subsequent suspected regurgitation.

In one or more embodiments, the automatically determining the time of the suspected regurgitation and predicting the time of at least one subsequent suspected regurgitation comprises:
acquiring electrocardiogram data while performing the ultrasound imaging, using the electrocardiogram data to determine a cardiac cycle, and determining a time range of the suspected regurgitation in a complete cardiac cycle; and
predicting the at least one suspected regurgitation in at least one subsequent cardiac cycle based on the time range of the suspected regurgitation in a complete cardiac cycle.

In one or more embodiments, the cardiac cycle and the prediction of the at least one suspected regurgitation are displayed in real time.

In one or more embodiments, an electrocardiogram waveform of the cardiac cycle is displayed in real time, and an electrocardiogram waveform within the time range of the at least one suspected regurgitation is highlighted.

In one or more embodiments, one of a plurality of imaging modes is selected in advance as an imaging mode of the second ultrasound imaging in response to an operation by a user.

In one or more embodiments, the selected one of the imaging modes is switched for the second ultrasound imaging in response to an operation by the user.

According to an aspect of the embodiments of the present application, an ultrasound imaging apparatus is provided. The ultrasound imaging apparatus comprises a memory and a processor. The memory stores a computer program, and the processor is configured to execute the computer program to implement the ultrasound imaging method described in the previous aspect.

According to an aspect of the embodiments of the present application, a computer program product is provided. The computer program product comprises at least a computer program, and when the computer program is executed by a processor, the ultrasound imaging method described in the previous aspect is executed.

One of the beneficial effects of the embodiments of the present application is that: the suspected regurgitation in the two-dimensional color flow image is automatically identified, and the second ultrasound imaging is automatically performed based on the identification result of the suspected regurgitation, to generate the second ultrasound image containing the suspected regurgitation region, the second ultrasound image containing the blood flow information and being different from the two-dimensional color flow image. Therefore, a workload of the physician can be reduced. More importantly, suspected regurgitation is automatically determined and secondary imaging is automatically performed, to improve accuracy of detecting cardiac regurgitation and increase diagnostic confidence of the physician.

With reference to the following description and drawings, specific implementations of the embodiments of the present application are disclosed in detail, and the way in which the principles of the embodiments of the present application can be employed are illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of an ultrasound imaging method according to embodiments of the present application;

FIG. 2 shows a schematic diagram of a generated two-dimensional color flow image of a heart;

FIG. 3 is a schematic diagram of regurgitation detection according to embodiments of the present application;

FIG. 4 is a schematic diagram of a second ultrasound image according to embodiments of the present application;

FIG. 5 is a schematic diagram of alignment of suspected regurgitation locations across different modes according to embodiments of the present application;

FIG. 6 is a schematic diagram of alignment of suspected regurgitation occurrence times across different modes according to embodiments of the present application;

FIG. 7 is a schematic diagram of an interaction interface of an ultrasound imaging apparatus according to embodiments of the present application;

FIG. 8 is another schematic diagram of an interaction interface of an ultrasound imaging apparatus according to embodiments of the present application;

FIG. 9 is another schematic diagram of an interaction interface of an ultrasound imaging apparatus according to embodiments of the present application; and

FIG. 10 is a schematic diagram of an ultrasound imaging system according to embodiments of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements with respect to naming, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies. Similar terms such as “connect”, “link”, and “couple” used in the embodiments of the present application are not limited to physical or mechanical connections, but may include electrical connections, whether direct or indirect.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “based on” should be construed as “based at least in part on . . . ”, unless otherwise specified in the context.

The features described and/or illustrated for one implementation may be used in one or more other implementations in the same or similar way, be combined with features in other embodiments, or replace features in other implementations. The terms “include/comprise” when used herein refer to the presence of features, integrated components, steps, or assemblies, but do not preclude the presence or addition of one or more other features, integrated components, steps, or assemblies.

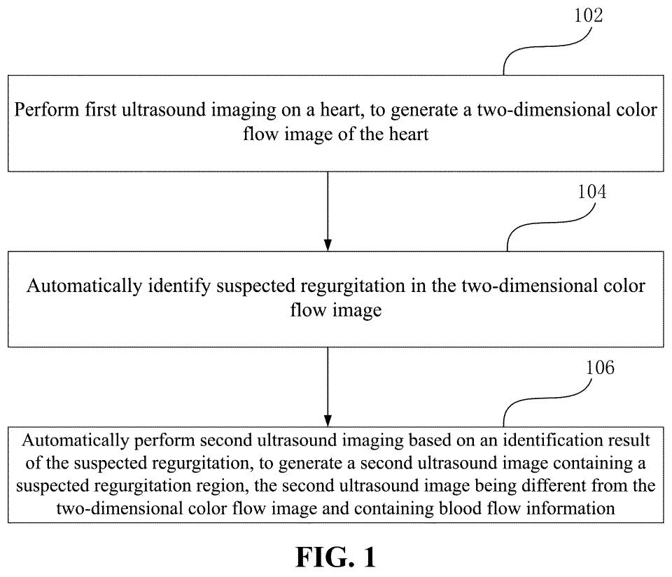

The embodiments of the present application provide an ultrasound imaging method for assisting in determining cardiac regurgitation. FIG. 1 is a schematic diagram of the ultrasound imaging method according to the embodiments of the present application. As shown in FIG. 1, the method includes:
102, performing first ultrasound imaging on a heart, to generate a two-dimensional color flow image of the heart;
104, automatically identifying suspected regurgitation in the two-dimensional color flow image; and
106, automatically performing second ultrasound imaging based on an identification result of the suspected regurgitation, to generate a second ultrasound image containing a suspected regurgitation region, the second ultrasound image being different from the two-dimensional color flow image and containing blood flow information.

An ultrasound imaging system may perform imaging on a tissue or an organ to be examined in a plurality of modes including, for example, a color flow (CF) mode, a continuous wave Doppler (CW) mode, a pulsed wave Doppler (PW) mode, and a four-dimensional (4D) mode.

In the color flow mode, a two-dimensional color picture is obtained, which can display the direction and speed of a blood flow to implement dynamic display of the blood flow, and one two-dimensional color picture is one section of a three-dimensional image. In the continuous wave Doppler mode, the ultrasound imaging system makes use of two independent sensor components, one of which transmits a continuous signal and the other of which monitors a reflected echo signal. A signal received in the continuous wave Doppler mode is a blood flow signal on an entire line. Because a sampling rate is high, aliasing does not occur in frequency spectra, and the frequency spectra are usually used to detect a high-speed blood flow in the heart, so that the pulsed wave Doppler mode is usually supported in a heart probe, and obtained flow rates on the frequency spectra represent blood flow rates on the entire line. In the pulsed wave Doppler, assessment is not performed on a blood flow in an entire lumen but rather focuses on measuring an instantaneous blood flow frequency spectrum at a certain small region. In this mode, signals are transmitted according to a certain time rule, a received signal is correlated with the depth of a sampling gate (usually indicated by two small horizontal lines) and is just a blood flow frequency offset signal in the sampling gate. Due to a limited sampling rate, when the blood flow rate is high, aliasing may occur in frequency spectra. All probes, whether cardiac or non-cardiac, support the pulsed wave Doppler mode. An obtained flow rate on a frequency spectrum represents a blood flow rate in the sampling gate (usually indicated by two small horizontal lines). The four-dimensional mode may also be referred to as a real-time three-dimensional mode, i.e., including a three-dimensional space and a time dimension. In this mode, a real-time three-dimensional image of an observed object may be obtained.

In addition, the ultrasound imaging system may further include other modes, for example, an A-mode, a B-mode, and an M-mode, for which reference can be made to the related art, and are not limited in the present application.

In the embodiments of the present application, in 102, ultrasound imaging may be performed on the heart by using an existing technique to generate a two-dimensional color flow image of the heart. For example, ultrasound imaging may be performed on the heart in the CF mode to generate a two-dimensional color flow image of the heart. However, the present application is not limited thereto, and another mode in which a two-dimensional color flow image of the heart may be generated may also be used for the processing in 102.

In the embodiments of the present application, in 102, a plurality of two-dimensional color flow images of the heart may be generated, and different two-dimensional color flow images may be different sections of a three-dimensional image corresponding to the heart.

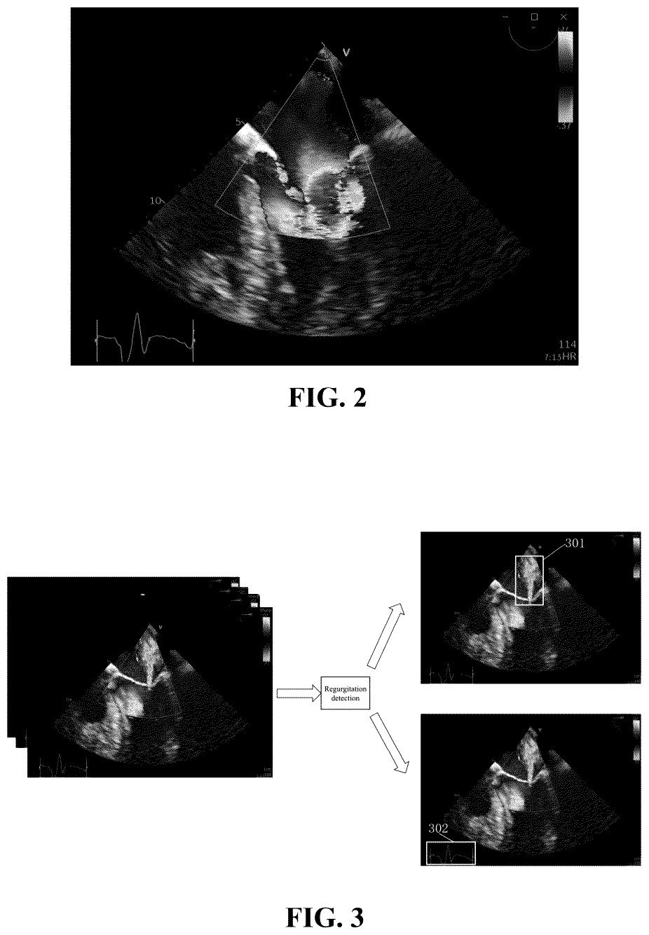

FIG. 2 shows a schematic diagram of a two-dimensional color flow image of a heart generated by means of 102. As shown in FIG. 2, different objects, including tissues and blood flows, are shown in different colors in the two-dimensional color flow image.

In the embodiments of the present application, in 104, suspected regurgitation in the two-dimensional color flow image is automatically identified. That is, the ultrasound imaging system analyzes an obtained two-dimensional color image to automatically identify suspected regurgitation, and a physician does not need to determine whether suspected regurgitation is occurring in the CF mode.

In current common cardiac regurgitation detection, the physician needs to observe two-dimensional color flow images to identify suspected regurgitation. However, the number of two-dimensional color images is usually large. After entering the CF mode, the physician needs to wait for generation of two-dimensional color flow images, and observe and identify the generated two-dimensional color flow images one by one, which requires the physician to spend a great deal of time and effort to identify suspected regurgitation in the two-dimensional color flow images. Therefore, a heavy workload is imposed on the physician, and missed identification easily occurs.

However, in the embodiments of the present application, identification is performed on the two-dimensional color flow image by the ultrasound imaging system itself, and even if the number of two-dimensional color flow images is large, efficient identification can be implemented by the ultrasound imaging system.

In the embodiments of the present application, for the automatic identification of suspected regurgitation in the two-dimensional color flow image by the ultrasound imaging system, reference may be made to existing techniques. For example, automatic identification may be performed on a systolic period of the heart, and it may be determined whether a blood flow signal, etc., from a ventricle to an atrium can be detected during the systolic period. Alternatively, the identification may be implemented by means of artificial intelligence, for example, automatic determination is performed by using a trained neural network. Alternatively, reference may be made to any other prior art in the art, including, but not limited to, patent U.S. Ser. No. 16/154,202, etc. It should be understood that the above description is intended to be illustrative, and another existing method for automatically identifying suspected regurgitation in a two-dimensional color flow image may also be used, which is not limited in the present application.

According to the foregoing embodiments, the suspected regurgitation in the two-dimensional color flow image is automatically identified, and the second ultrasound imaging is automatically performed based on the identification result of the suspected regurgitation, to generate the second ultrasound image containing the suspected regurgitation region, the second ultrasound image containing the blood flow information and being different from the two-dimensional color flow image. Therefore, the workload of the physician can be reduced, and the accuracy of detecting cardiac regurgitation can be improved.

For example, in an existing technical solution for detecting regurgitation, a physician needs to rely on his/her experience to identify regurgitation according to a two-dimensional color flow image. However, in the embodiments of the present application, the suspected regurgitation in the two-dimensional color flow image can be automatically identified by an ultrasound imaging apparatus, thereby reducing the workload of the physician and improving the accuracy and efficiency of detection. In addition, in an existing regurgitation detection technique, when a physician identifies that a suspected blood flow is occurring, the physician needs to manually switch to a Doppler mode or 4D mode. After switching to the Doppler mode or 4D mode, the physician needs to rely on his/her experience to identify, according to an identification result of the two-dimensional color flow image, regurgitation in an image that is in the Doppler mode or 4D mode. For example, the physician manually moves a cursor or sets a region of interest (ROI) in the image that is in the Doppler mode or 4D mode, to further perform regurgitation detection. However, in the embodiments of the present application, the second ultrasound imaging is automatically performed based on the identification result obtained through automatic identification, and the second ultrasound image containing the suspected regurgitation region and the blood flow information is generated. In this way, even if cross-mode ultrasound imaging is performed, the physician only needs to focus on a relevant region in the second ultrasound image to perform regurgitation detection, thereby significantly reducing the workload of the physician and improving the accuracy of regurgitation detection.

In addition, in an entire process of regurgitation detection, an ultrasound probe needs to remain stationary. However, in conventional detection, a physician typically needs to hold the ultrasound probe with one hand while using the other hand to perform operations such as mode switching and cursor moving. In this process, it is difficult to keep a location of the ultrasound probe unchanged at all times, the ultrasound probe may generate a positional offset, but regurgitation detection has very high spatial and temporal sensitivity, that is, a time window and a location region at which regurgitation is occurring are both small. Therefore, even if the physician identifies suspected regurgitation in the CF mode, a slight offset of the ultrasound probe in the process of detection may result in missed identification of an occurrence location of regurgitation in the Doppler mode or 4D mode. However, in the embodiments of the present application, the physician does not need to perform operations such as mode switching and cursor moving, which can improve the accuracy of regurgitation detection.

In one or more embodiments, the second ultrasound imaging includes at least one of continuous wave Doppler imaging, pulsed wave Doppler imaging, and four-dimensional color ultrasound imaging. Compared to a single CF mode, integrating continuous wave Doppler imaging, pulsed wave Doppler imaging, and four-dimensional color ultrasound imaging for comprehensive determination enables more accurate regurgitation detection. Therefore, the accuracy of regurgitation detection can be improved. However, the present application is not limited thereto, and the second ultrasound imaging may also be another imaging mode in which regurgitation detection can be performed.

In the present application, the second ultrasound imaging may be one of the three of continuous wave Doppler imaging, pulsed wave Doppler imaging, and four-dimensional color ultrasound imaging, or the second ultrasound imaging may include any two or all of the three. For example, the second ultrasound imaging includes continuous wave Doppler imaging and four-dimensional color ultrasound imaging. After automatically identifying the suspected regurgitation in the two-dimensional color flow image, the ultrasound imaging system automatically enters a continuous wave Doppler imaging mode for regurgitation detection, and then automatically enters a four-dimensional color ultrasound imaging mode for further regurgitation detection.

In one or more embodiments, in 104, occurrence of the suspected regurgitation in the two-dimensional color flow image is automatically identified and a location of the suspected regurgitation is automatically determined.

That is, the ultrasound imaging apparatus can automatically determine the location of the suspected regurgitation according to the two-dimensional color flow image. Therefore, the second ultrasound image can be generated by using the determined location information of the suspected regurgitation, so that the physician performs regurgitation detection and does not need to pay attention to the second ultrasound image that is not related to the location information of the suspected regurgitation in the second ultrasound imaging, which can improve the efficiency and accuracy of regurgitation detection.

In one or more embodiments, in 106, the automatically performing second ultrasound imaging based on an identification result of the suspected regurgitation includes: automatically controlling, based on the determined location of the suspected regurgitation, an imaging parameter of the second ultrasound imaging, to correlate the second ultrasound imaging with the location of the suspected regurgitation.

For example, by controlling the imaging parameter of the second ultrasound imaging, the second ultrasound image generated by the second ultrasound imaging includes the location where the suspected regurgitation is occurring. Alternatively, in another example, the imaging parameter of the second ultrasound imaging may be further configured for the location information of the suspected regurgitation to improve the image quality of the location of the suspected regurgitation. Therefore, there is no need to pay attention to the second ultrasound image that is not related to the location of the suspected regurgitation in the second ultrasound imaging, which can improve the efficiency and accuracy of regurgitation detection.

In one or more embodiments, in 106, the second ultrasound imaging may include continuous wave Doppler imaging or pulsed wave Doppler imaging; and the automatically controlling an imaging parameter of the second ultrasound imaging includes: automatically positioning, based on the location of the suspected regurgitation, a location of a sampling window of the second ultrasound imaging, so that the sampling window passes through the suspected regurgitation region; and automatically performing the second ultrasound imaging on the location of the sampling window, to generate continuous wave Doppler imaging or pulsed wave Doppler imaging related to the suspected regurgitation region. In this way, in the second ultrasound imaging, only the continuous wave Doppler imaging or the pulsed wave Doppler imaging of the sampling window passing through the suspected regurgitation region and the second ultrasound image that is not related to the location of the suspected regurgitation are generated, thereby improving the efficiency and accuracy of regurgitation detection.

In one or more embodiments, in 106, the second ultrasound imaging includes four-dimensional color ultrasound imaging; and the automatically controlling an imaging parameter of the second ultrasound imaging includes: generating a four-dimensional color ultrasound image of the heart, mapping the location of the suspected regurgitation in the two-dimensional color flow image into the four-dimensional color ultrasound image, and highlighting the location.

In this way, in the four-dimensional color ultrasound imaging as the second ultrasound imaging, the location of the suspected regurgitation in the two-dimensional color flow image is mapped into the four-dimensional color ultrasound image and is highlighted. The physician can quickly determine a region that needs to be focused on in the four-dimensional color ultrasound image, which can improve the efficiency and accuracy of regurgitation detection.

In one or more embodiments, the imaging method further includes: simultaneously displaying the two-dimensional color flow image, the location of the suspected regurgitation in the two-dimensional color flow image, the four-dimensional color ultrasound image, and the location of the suspected regurgitation in the four-dimensional color ultrasound image. That is, in a display apparatus of the ultrasound imaging apparatus, ultrasound images in different modes and a location where suspected regurgitation is occurring in each image are simultaneously displayed. Therefore, it is convenient for the physician to perform comparison and analysis, the accuracy of regurgitation detection can be improved, and the confidence of the physician in a detection result can be increased.

In one or more embodiments, in 102, the automatically identifying suspected regurgitation in the two-dimensional color flow image further includes: automatically determining the time of the suspected regurgitation and predicting the time of at least one subsequent suspected regurgitation.

In different cardiac cycles, it is usually believed that the time when regurgitation occurs is constant at time points or during time periods in the cardiac cycles. In this way, by determining the time when the suspected regurgitation occurs in the two-dimensional color flow image, for example, the time when the regurgitation occurs at the time points or during the time periods in the cardiac cycles, the time when suspected regurgitation will subsequently occur can be predicted. Therefore, the physician can be further alerted to the second ultrasound image that needs to be focused on, that is, the physician only needs to focus on the second ultrasound image during a time period during which suspected regurgitation is about to occur, interference of an irrelevant image on a detection result can be reduced, and the efficiency and accuracy of regurgitation detection can be improved.

In one or more embodiments, the automatically determining the time of the suspected regurgitation and predicting the time of at least one subsequent suspected regurgitation includes: acquiring electrocardiogram data while performing the ultrasound imaging, using the electrocardiogram data to determine a cardiac cycle, and determining a time range of the suspected regurgitation in a complete cardiac cycle; and predicting the at least one suspected regurgitation in at least one subsequent cardiac cycle based on the time range of the suspected regurgitation in a complete cardiac cycle. Therefore, the time when suspected regurgitation will subsequently occur can be predicted.

However, the present application is not limited thereto. For example, a timing component may be provided, and in the process of automatically identifying the suspected regurgitation in the two-dimensional color flow image, the timing component records a time interval between two adjacent occurrences of regurgitation, so that the time when suspected regurgitation will subsequently occur can be predicted.

In one or more embodiments, the cardiac cycle and the prediction of the at least one suspected regurgitation are displayed in real time. For example, an electrocardiogram waveform of the cardiac cycle is displayed in real time, and the electrocardiogram waveform in the time range of the at least one suspected regurgitation is highlighted. However, the present application is not limited thereto. For example, a time interval that is represented by a straight line segment and that corresponds to the duration of the cardiac cycle may be displayed in real time. In this way, the physician can be alerted to the time when suspected regurgitation is imminent, so that the physician can increase his/her attention, thereby improving the efficiency and accuracy of regurgitation detection.

Illustrative descriptions are provided below with reference to the drawings.

FIG. 3 is a schematic diagram of regurgitation detection according to embodiments of the present application. As shown in FIG. 3, for a first ultrasound image generated in a first mode, the first ultrasound image includes a plurality of frame images, for example, includes at least ultrasound images during one scanning period. Regurgitation detection is performed on the generated first ultrasound image to automatically identify suspected regurgitation in a two-dimensional color flow image, including a location where a suspected blood flow is occurring shown in 301 and the time 302 when the suspected blood flow occurs shown in 302. The time may be a time location in one cardiac cycle shown in FIG. 3.

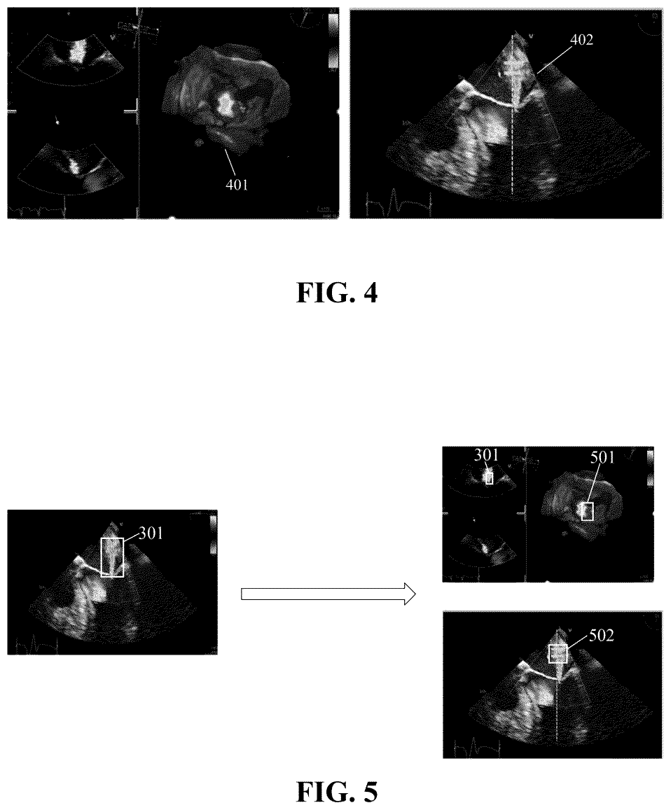

FIG. 4 is a schematic diagram of a second ultrasound image according to embodiments of the present application. As shown in FIG. 4, a 4D image 401 and a CW image 402 are automatically and sequentially generated based on an identification result of the suspected regurgitation in FIG. 3, the 4D image 401 and the CW image 402 being different from the two-dimensional color flow image shown in FIG. 3. It should be noted that although FIG. 4 shows the 4D image and the CW image, the present application is not limited thereto. The second ultrasound image may include only the 4D image or the CW image, or may be another image, for example, a PW image. In addition, as shown in FIG. 4, the 4D image 401 may also include the two-dimensional color flow image for comparison with the 4D image, to facilitate a physician in identifying regurgitation.

FIG. 5 is a schematic diagram of alignment of suspected regurgitation locations across different modes according to embodiments of the present application. As shown in FIG. 5, according to the detected location of the suspected regurgitation shown by a reference numeral 301 in the first ultrasound image in a first ultrasound imaging mode, the location where the suspected regurgitation is occurring as shown by 501 is marked in the 4D image and a sampling window is marked with a pair of horizontal lines in the CW image as shown by 502. It should be noted that FIG. 5 shows a representation of the sampling window in the CW mode. However, in the CW mode, the sampling window may also refer to an entire sampling line shown in FIG. 5, without a pair of horizontal lines. In addition, although not shown, the PW mode may also be used and the verified sampling window may be positioned.

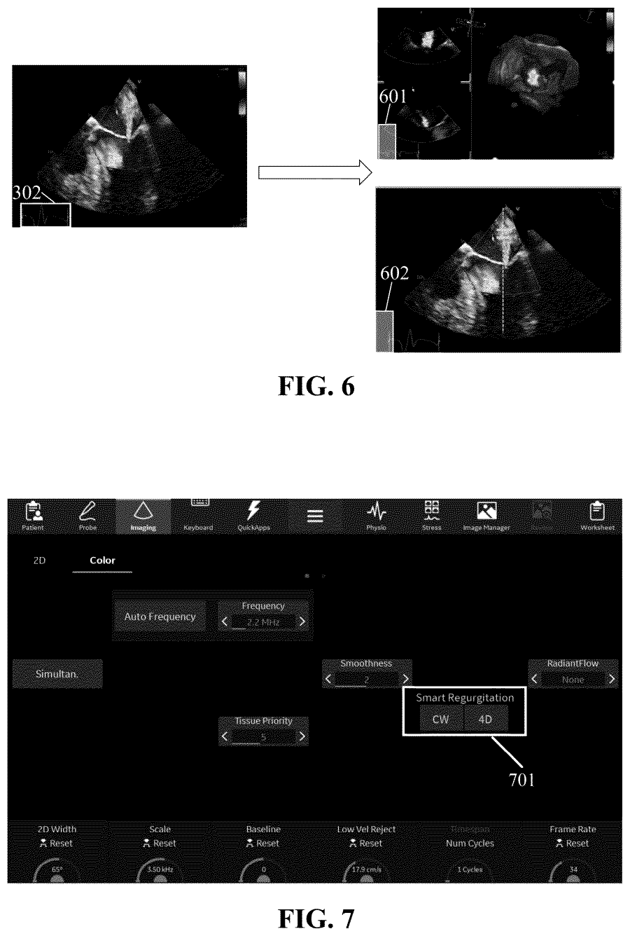

FIG. 6 is a schematic diagram of alignment of suspected regurgitation occurrence times across different modes according to embodiments of the present application. As shown in FIG. 6, according to the time when suspected regurgitation detected in a first ultrasound image in a first ultrasound imaging mode occurs, for example, a time location of the suspected regurgitation in one cardiac cycle, a cardiac cycle represented by an electrocardiogram waveform is displayed in a 4D image and a CW image, and a time range during which the suspected regurgitation occurs is highlighted in red as shown by 601 and 602 on the cardiac cycle.

Therefore, a physician can detect regurgitation according to the second ultrasound images shown in FIG. 5 and FIG. 6, thereby performing regurgitation diagnosis.

In one or more embodiments, one of a plurality of imaging modes is selected in advance as an imaging mode of the second ultrasound imaging in response to an operation by a user. That is, a plurality of imaging modes may be displayed as a second ultrasound imaging mode on a display component such as a display screen of an ultrasound imaging apparatus, and when the user needs to perform cardiac regurgitation detection, for example, before the first ultrasound imaging, one of the imaging modes may be selected in advance as the imaging mode of the second ultrasound imaging, and then the ultrasound imaging apparatus may automatically enter the second ultrasound imaging mode selected by the user after the completion of the first ultrasound imaging operation. Therefore, the user does not need to perform a mode switching operation in the entire process of the cardiac regurgitation detection, and may focus on observing the two-dimensional color flow image and the second ultrasound image to identify regurgitation, thereby improving the accuracy of regurgitation detection.

In one or more embodiments, the selected one of the imaging modes is switched for the second ultrasound imaging in response to an operation by the user. For example, after the end of one second ultrasound imaging, for example, the end of the CW mode, the user may further operate to switch to another second ultrasound imaging mode, for example, switch to a 4D mode, to perform the second ultrasound imaging again. Therefore, diversified second ultrasound imaging can be flexibly provided to further improve the accuracy of regurgitation detection. The embodiments of the present specification further provide an ultrasound imaging apparatus. The apparatus includes: a memory and a processor, the memory storing a computer program, and the processor being configured to execute the computer program to implement the ultrasound imaging method described in the foregoing embodiments. For the ultrasound imaging method, refer to the above description, which will not be repeated herein. The foregoing memory may include, for example, a ROM, a floppy disk, a hard disk, an optical disk, a magneto-optical disk, a CD-ROM, or a non-volatile memory card.

In the embodiments of the present application, the ultrasound imaging apparatus may further include a display apparatus. The display apparatus may be used as a human-computer interaction interface, and a dedicated operation assembly may be further provided in the interaction interface to allow a physician to perform one-click diagnosis of regurgitation.

FIG. 7 is a schematic diagram of an interaction interface of an ultrasound imaging apparatus according to embodiments of the present application.

As shown in FIG. 7, a smart regurgitation button may be provided in the interaction interface, and includes two sub-item buttons, i.e., a CW sub-item button and a 4D sub-item button. The smart regurgitation button receives user input when regurgitation detection is required. Specifically, the user may touch the CW sub-item button, after the user touches the CW sub-item button, the ultrasound imaging system automatically enters, for example, a CF mode to generate a two-dimensional color flow image, then automatically identifies suspected regurgitation in the two-dimensional color flow image, and automatically enters a CW mode based on an identification result of the suspected regurgitation in the CF mode to perform second ultrasound imaging, to generate a second ultrasound image containing a suspected regurgitation region, and then, a physician may perform regurgitation detection by using the second ultrasound image. In addition, the user may also touch the 4D sub-item button, after the user touches the 4D sub-item button, the ultrasound imaging system automatically enters, for example, the CF mode to generate a two-dimensional color flow image, then automatically identifies suspected regurgitation in the two-dimensional color flow image, and automatically enters a 4D mode based on an identification result of the suspected regurgitation in the CF mode to perform second ultrasound imaging, to generate a second ultrasound image containing a suspected regurgitation region, and then, the physician may perform regurgitation detection by using the second ultrasound image.

In some embodiments, the interaction interface of the ultrasound imaging apparatus may further include a return button and a switch button, allowing the user to return to a first ultrasound imaging mode or enter another ultrasound imaging mode when the user has entered a second ultrasound imaging mode.

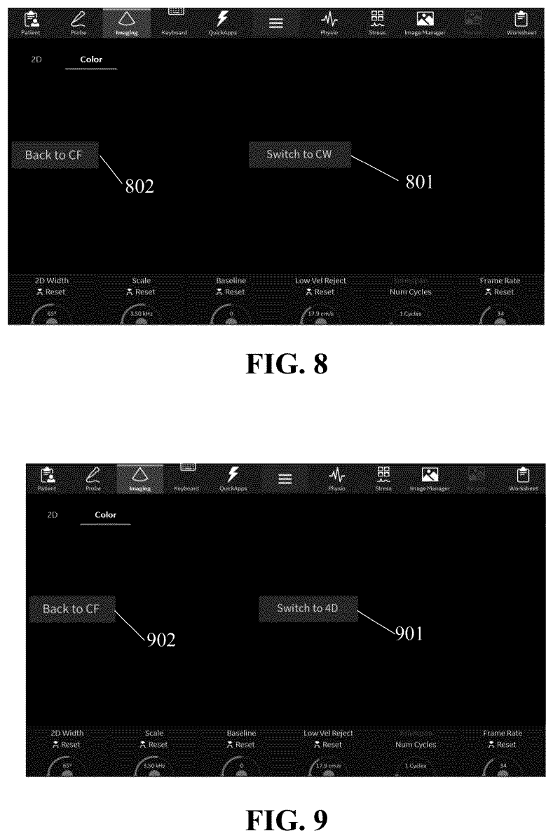

For example, FIG. 8 and FIG. 9 are other schematic diagrams of an interaction interface of an ultrasound imaging apparatus according to embodiments of the present application. As shown in FIG. 8, the interaction interface of the ultrasound imaging apparatus may further include a return button 802 “Back to CF” and a switch button 801 “Switch to CW”. In this way, when regurgitation detection in 4D ultrasound imaging is completed, the user can select to return to the CF mode or switch to the CW mode to further perform regurgitation detection, thereby improving the flexibility of regurgitation detection.

As shown in FIG. 9, the interaction interface of the ultrasound imaging apparatus may further include a return button 902 “Back to CF” and a switch button 901 “Switch to 4D”. In this way, when regurgitation detection in CW ultrasound imaging is completed, the user can select to return to the CF mode or switch to the 4D mode to further perform regurgitation detection, thereby improving the flexibility of regurgitation detection.

It should be noted that what are shown in FIG. 7 to FIG. 9 are merely illustrative descriptions of a user interface regarding regurgitation detection, and the present application is not limited thereto.

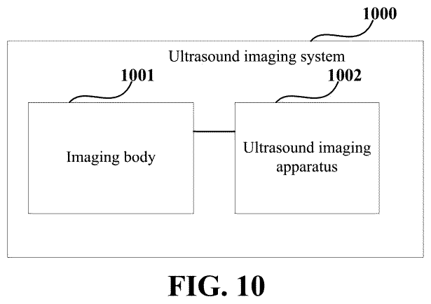

The embodiments of the present application further provide an ultrasound imaging system. FIG. 10 is a schematic diagram of an ultrasound imaging system according to embodiments of the present application. As shown in FIG. 10, the system 1000 may include an imaging body 1001 and an ultrasound imaging apparatus 1002 connected to the imaging body 1001. The imaging body 1001 may include various components for probing a human body, such as an ultrasound probe. The ultrasound imaging apparatus 1002 is configured to control the imaging body 1001 to execute the ultrasound imaging method in the foregoing embodiments.

In some embodiments, the ultrasound imaging apparatus may be configured separately from a controller of the ultrasound imaging system. For example, the ultrasound imaging apparatus is configured as a chip, etc., connected to the controller of the ultrasound imaging system, and the two may control each other. Alternatively, functions of the ultrasound imaging apparatus may be integrated into the controller of the ultrasound imaging system. This is not limited in the embodiments of the present application.

The ultrasound imaging system may further include other structural components not shown in the figure. For example, the ultrasound imaging system may further include a carrier for supporting and carrying the ultrasound imaging apparatus. In addition, reference may be made to the related art. This is not limited in the embodiments of the present application.

The embodiments of the present application further provide a computer-readable program, where the program, when executed in an ultrasound imaging apparatus or an imaging system, causes a computer to execute, in the apparatus or the imaging system, the ultrasound imaging method described in the foregoing embodiments.

The embodiments of the present application further provide a storage medium having a computer-readable program stored thereon, where the computer-readable program causes a computer to execute, in an apparatus or an imaging system, the ultrasound imaging method described in the foregoing embodiments.

The embodiments of the present application further provide a computer program product. The computer program product includes at least a computer program. When the computer program is executed by a processor, the ultrasound imaging method described in the foregoing embodiments is executed.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may correspond to the steps shown in the figures, respectively. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and the variations and modifications also fall within the scope of the present application.

1. An ultrasound imaging method for assisting in determining cardiac regurgitation, comprising:
performing first ultrasound imaging on a heart, to generate a two-dimensional color flow image of the heart;
automatically identifying suspected regurgitation in the two-dimensional color flow image; and
automatically performing second ultrasound imaging based on an identification result of the suspected regurgitation, to generate a second ultrasound image containing a suspected regurgitation region, the second ultrasound image being different from the two-dimensional color flow image and containing blood flow information.

2. The method according to claim 1, wherein the automatically identifying suspected regurgitation in the two-dimensional color flow image comprises:
automatically identifying occurrence of the suspected regurgitation in the two-dimensional color flow image, and automatically determining a location of the suspected regurgitation.

3. The method according to claim 2, wherein the automatically performing second ultrasound imaging based on an identification result of the suspected regurgitation comprises:
automatically controlling, based on the determined location of the suspected regurgitation, an imaging parameter of the second ultrasound imaging, to correlate the second ultrasound imaging with the location of the suspected regurgitation.

4. The method according to claim 1, wherein
the second ultrasound imaging comprises at least one of continuous wave Doppler imaging, pulsed wave Doppler imaging, and four-dimensional color ultrasound imaging.

5. The method according to claim 3, wherein the second ultrasound imaging comprises continuous wave Doppler imaging or pulsed wave Doppler imaging; and the automatically controlling an imaging parameter of the second ultrasound imaging comprises:
automatically positioning, based on the location of the suspected regurgitation, a location of a sampling window of the second ultrasound imaging, so that the sampling window passes through the suspected regurgitation region; and automatically performing the second ultrasound imaging on the location of the sampling window, to generate a continuous wave Doppler image or a pulsed wave Doppler image related to the suspected regurgitation region.

6. The method according to claim 3, wherein the second ultrasound imaging comprises four-dimensional color ultrasound imaging; and the automatically controlling an imaging parameter of the second ultrasound imaging comprises:
generating a four-dimensional color ultrasound image of the heart, mapping the location of the suspected regurgitation in the two-dimensional color flow image into the four-dimensional color ultrasound image, and highlighting the location.

7. The method according to claim 6, further comprising:
simultaneously displaying the two-dimensional color flow image, the location of the suspected regurgitation in the two-dimensional color flow image, the four-dimensional color ultrasound image, and the location of the suspected regurgitation in the four-dimensional color ultrasound image.

8. The method according to claim 2, wherein the automatically identifying suspected regurgitation in the two-dimensional color flow image further comprises:
automatically determining the time of the suspected regurgitation and predicting the time of at least one subsequent suspected regurgitation.

9. The method according to claim 8, wherein the automatically determining the time of the suspected regurgitation and predicting the time of at least one subsequent suspected regurgitation comprises:
acquiring electrocardiogram data while performing the ultrasound imaging, using the electrocardiogram data to determine a cardiac cycle, and determining a time range of the suspected regurgitation in a complete cardiac cycle; and
predicting the at least one suspected regurgitation in at least one subsequent cardiac cycle based on the time range of the suspected regurgitation in a complete cardiac cycle.

10. The method according to claim 9, further comprising:
displaying the cardiac cycle and the prediction of the at least one suspected regurgitation in real time.

11. The method according to claim 10, wherein the displaying in real time comprises:
displaying an electrocardiogram waveform of the cardiac cycle in real time, and highlighting an electrocardiogram waveform within the time range of the at least one suspected regurgitation.

12. The method according to claim 1, further comprising:
selecting, in response to an operation by a user, one of a plurality of imaging modes in advance as an imaging mode of the second ultrasound imaging.

13. The method according to claim 12, further comprising:
switching, in response to an operation by the user, the selected one of the imaging modes for the second ultrasound imaging.

14. An ultrasound imaging system, comprising:
a memory storing instructions; and
a processor configured to execute the instructions to:
perform first ultrasound imaging on a heart, to generate a two-dimensional color flow image of the heart;
automatically identify suspected regurgitation in the two-dimensional color flow image; and
automatically perform second ultrasound imaging based on an identification result of the suspected regurgitation, to generate a second ultrasound image containing a suspected regurgitation region, the second ultrasound image being different from the two-dimensional color flow image and containing blood flow information.

15. The method according to claim 14, wherein the automatically identifying suspected regurgitation in the two-dimensional color flow image comprises:
automatically identifying occurrence of the suspected regurgitation in the two-dimensional color flow image, and automatically determining a location of the suspected regurgitation.

16. The method according to claim 15, wherein the automatically performing second ultrasound imaging based on an identification result of the suspected regurgitation comprises:
automatically controlling, based on the determined location of the suspected regurgitation, an imaging parameter of the second ultrasound imaging, to correlate the second ultrasound imaging with the location of the suspected regurgitation.

17. The method according to claim 14, wherein
the second ultrasound imaging comprises at least one of continuous wave Doppler imaging, pulsed wave Doppler imaging, and four-dimensional color ultrasound imaging.

18. The method according to claim 17, wherein the second ultrasound imaging comprises continuous wave Doppler imaging or pulsed wave Doppler imaging; and the automatically controlling an imaging parameter of the second ultrasound imaging comprises:
automatically positioning, based on the location of the suspected regurgitation, a location of a sampling window of the second ultrasound imaging, so that the sampling window passes through the suspected regurgitation region; and automatically performing the second ultrasound imaging on the location of the sampling window, to generate a continuous wave Doppler image or a pulsed wave Doppler image related to the suspected regurgitation region.

19. The system according to claim 15, wherein the automatically identifying suspected regurgitation in the two-dimensional color flow image further comprises:
automatically determining the time of the suspected regurgitation and predicting the time of at least one subsequent suspected regurgitation.

20. A non-transitory computer-readable medium, having a computer program stored therein, wherein the computer program has at least one code segment, the at least one code segment being executable by a machine to cause the machine to perform the steps of:
performing first ultrasound imaging on a heart, to generate a two-dimensional color flow image of the heart;
automatically identifying suspected regurgitation in the two-dimensional color flow image; and
automatically performing second ultrasound imaging based on an identification result of the suspected regurgitation, to generate a second ultrasound image containing a suspected regurgitation region, the second ultrasound image being different from the two-dimensional color flow image and containing blood flow information.
